(19) United States
(12) Patent Application Publication
(10) Pub. No.: US 2026/0013867 A1
(43) Pub. Date: Jan. 15, 2026

(54) LEFT ATRIAL APPENDAGE CLOSURE DEVICE

(71) Applicant: BOSTON SCIENTIFIC SCIMED, INC., Maple Grove, MN (US)

(72) Inventors: Jerry Sy-Shyan Wang, Edina, MN (US); Philip Andrew Litecky, Forest Lake, MN (US); Timothy Hillukka, Monticello, MN (US); Graham Krumpelmann, Stillwater, MN (US); Joshua David Tillman, New Brighton, MN (US)

(73) Assignee: BOSTON SCIENTIFIC SCIMED, INC., Maple Grove, MN (US)

(21) Appl. No.: 19/268,379

(22) Filed: Jul. 14, 2025

Related U.S. Application Data
(60) Provisional application No. 63/671,378, filed on Jul. 15, 2024.

(51) Int. Cl. A61B 17/12; A61B 17/00

(52) U.S. Cl. CPC A61B 17/12122; A61B 17/1214; A61B 2017/00367; A61B 2017/00867

(57) ABSTRACT

A left atrial appendage closure (LAAC) device is adapted to pull a left atrial appendage (LAA) closed on itself. The LAAC device includes a central hub and a plurality of engagement arms that are coupled to the central hub. At least some of the plurality of engagement arms include a tissue engaging region having one or more hooks that extend in a first circumferential direction. A drive mechanism is engaged with the central hub and is adapted to rotate the central hub in the first circumferential direction.

### CROSS REFERENCE TO RELATED APPLICATIONS

This application claims the benefit of priority of U.S. Provisional Application No. 63/671,378 filed Jul. 15, 2024, the entire disclosure of which is hereby incorporated by reference.

### TECHNICAL FIELD

The disclosure relates generally to medical devices and more particularly to medical devices that are adapted for closing a left atrial appendage closure device.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a left atrial appendage closure (LAAC) device that is adapted to pull a left atrial appendage (LAA) closed on itself. The LAAC device includes a central hub and a plurality of engagement arms that are coupled to the central hub. At least some of the plurality of engagement arms include a tissue engaging region having one or more hooks extending in a first circumferential direction. A drive mechanism is engaged with the central hub and is adapted to rotate the central hub in the first circumferential direction.

Alternatively or additionally, prior to engagement with tissue of the LAA, at least some of the plurality of engagement arms may have an expanded, post-delivery configuration in which at least some of the plurality of engagement arms may extend radially outwardly from the central hub.

Alternatively or additionally, prior to engagement with tissue of the LAA, the plurality of engagement arms may have an expanded post-delivery configuration in which each of the engagement arms may be adapted to bend in a second circumferential direction that is opposite of the first circumferential direction.

Alternatively or additionally, after engagement with tissue of the LAA, the plurality of engagement arms may each be adapted to bend further in the second circumferential direction.

Alternatively or additionally, the LAAC device may further include a proximal cap that extends over the central hub and the drive mechanism and that includes an outer surface.

Alternatively or additionally, the outer surface of the proximal cap may be adapted to seal against tissue of the LAA when the LAA is pulled down onto the outer surface of the proximal cap.

Alternatively or additionally, the plurality of engagement arms and the central hub may be formed as an integral shape memory frame.

Alternatively or additionally, the integral shape memory frame may include a Nitinol frame.

Alternatively or additionally, the drive mechanism may be adapted to be releasably secured to a delivery device.

Alternatively or additionally, each of the plurality of engagement arms may have the same length.

Another example may be found in a left atrial appendage closure (LAAC) device that is adapted to pull a left atrial appendage (LAA) closed on itself. The LAAC device includes a central hub and a plurality of engagement arms that, prior to engagement with tissue of the LAA, have an expanded, post-delivery configuration in which each of the plurality of engagement arms extend radially outwardly from the central hub. At least some of the plurality of engagement arms include one or more hooks within a distal region thereof extending in a first circumferential direction.

Alternatively or additionally, the LAAC device may further include a drive mechanism that is engaged with the central hub and is adapted to rotate the central hub in the first circumferential direction.

Alternatively or additionally, wherein prior to engagement with tissue of the LAA, the plurality of engagement arms may each be adapted to bend in a second circumferential direction that is opposite of the first circumferential direction.

Alternatively or additionally, after engagement with tissue of the LAA, the plurality of engagement arms may each be adapted to bend further in the second circumferential direction.

Alternatively or additionally, the plurality of engagement arms and the central hub may be formed as an integral Nitinol frame.

Alternatively or additionally, the LAAC device may further include a proximal cap that extends over the central hub and includes an outer surface that is adapted to seal against tissue of the LAA when the LAA is pulled down onto the outer surface of the proximal cap.

Another example may be found in a left atrial appendage closure (LAAC) device that is adapted to pull a left atrial appendage (LAA) closed on itself. The LAAC device includes a central hub, a drive mechanism that is engaged with the central hub, and a plurality of engagement arms having a configuration in which each of the plurality of engagement arms extend radially outwardly from the central hub in the absence of applied forces. At least some of the plurality of engagement arms include one or more hooks within a distal region thereof that extend in a first circumferential direction.

Alternatively or additionally, the plurality of engagement arms may each be adapted to bend further in the second direction after the one or more hooks engage tissue.

Alternatively or additionally, the plurality of engagement arms and the central hub include a Nitinol frame.

Alternatively or additionally, the LAAC device may further include a proximal cap that extends over the central hub and includes an outer surface that is adapted to seal against tissue of the LAA when the LAA is pulled down onto the outer surface of the proximal cap.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross-sectional view of an LAA (left atrial appendage);

FIG. 2 provides additional views of particular shapes of LAAs that can be difficult to implant a left atrial appendage closure (LAAC) device into;

FIG. 3 is a perspective view of an illustrative LAAC device;

FIG. 4 is an exploded perspective view of the illustrative LAAC device of FIG. 3;

FIG. 5 provides a schematic illustration of how the illustrative LAAC device of FIG. 3 functions to close an LAA;

FIG. 6 is a schematic view of the illustrative LAAC device of FIG. 3, showing how the LAAC device accommodates a non-circular LAA;

FIG. 7 provides a schematic view of how the illustrative LAAC device of FIG. 3 accommodates a non-circular LAA;

FIGS. 8, 9 and 10 are schematic illustrations showing how the LAA is drawn down onto several different example proximal caps (forming part of the illustrative LAAC device of FIG. 3).

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following figures illustrate selected components and/or arrangements of an implant for occluding the left atrial appendage, a system for occluding the left atrial appendage, and/or methods of using the implant and/or the system. It should be noted that in any given figure, some features may not be shown, or may be shown schematically, for simplicity. Additional details regarding some of the components of the implant and/or the system may be illustrated in other figures in greater detail. While discussed in the context of occluding the left atrial appendage, the implant and/or the system may also be used for other interventions and/or percutaneous medical procedures within a patient. Similarly, the devices and methods described herein with respect to percutaneous deployment may be used in other types of surgical procedures, as appropriate. For example, in some examples, the devices may be used in a non-percutaneous procedure. Devices and methods in accordance with the disclosure may also be adapted and configured for other uses within the anatomy.

In some instances, a left atrial appendage closure (LAAC) device is adapted to pull a left atrial appendage (LAA) closed on itself. The LAAC device includes a central hub and a plurality of engagement arms that are coupled to the central hub. At least some of the plurality of engagement arms include a tissue engaging region having one or more hooks that extend in a first circumferential direction. A drive mechanism is engaged with the central hub and is adapted to rotate the central hub in the first circumferential direction.

In some cases, prior to engagement with tissue of the LAA, at least some of the plurality of engagement arms may have an expanded, post-delivery configuration in which at least some of the plurality of engagement arms extend radially outwardly from the central hub. In some cases, prior to engagement with tissue of the LAA, the plurality of engagement arms may have an expanded post-delivery configuration in which each of the engagement arms are adapted to bend in a second direction that is opposite of the first direction. After engagement with tissue of the LAA, the plurality of engagement arms may each be adapted to bend further in the second direction.

In some cases, the LAAC device may further include a proximal cap that extends over the central hub and the drive mechanism. An outer surface of the proximal cap may be adapted to seal against tissue of the LAA when the LAA is pulled down onto the outer surface of the proximal cap. In some cases, the plurality of engagement arms and the central hub may be formed as an integral shape memory frame. As an example, the integral shape memory frame may include a Nitinol frame. In some cases, the delivery mechanism may be adapted to be releasably secured to a delivery device. In some cases, each of the plurality of engagement arms may each be the same length.

In some instances, a left atrial appendage closure (LAAC) device is adapted to pull a left atrial appendage (LAA) closed on itself. The LAAC device includes a central hub and a plurality of engagement arms that, prior to engagement with tissue of the LAA, have an expanded, post-delivery configuration in which each of the plurality of engagement arms extend radially outwardly from the central hub. At least some of the plurality of engagement arms include one or more hooks within a distal region thereof extending in a first circumferential direction.

In some cases, the LAAC device may further include a drive mechanism that is engaged with the central hub and is adapted to rotate the central hub in the first circumferential direction. Prior to engagement with tissue of the LAA, the plurality of engagement arms may each be adapted to bend in a second direction that is opposite of the first direction. After engagement with tissue of the LAA, the plurality of engagement arms may each be adapted to bend further in the second direction. In some cases, the plurality of engagement arms and the central hub may be formed as an integral Nitinol frame. In some cases, the LAAC device may further include a proximal cap that extends over the central hub. The proximal cap includes an outer surface that is adapted to seal against tissue of the LAA when the LAA is pulled down onto the outer surface of the proximal cap.

In some instances, a left atrial appendage closure (LAAC) device is adapted to pull a left atrial appendage (LAA) closed on itself. The LAAC device includes a central hub, a drive mechanism that is engaged with the central hub, and a plurality of engagement arms having a configuration in which each of the plurality of engagement arms extend radially outwardly from the central hub in the absence of applied forces. At least some of the plurality of engagement arms include one or more hooks within a distal region thereof extending in a first circumferential direction.

In some cases, the plurality of engagement arms may each be adapted to bend further in the second direction after the one or more hooks engage tissue. In some cases, the plurality of engagement arms and the central hub may include a Nitinol frame. In some cases, the LAAC device may further include a proximal cap that extends over the central hub. The proximal cap includes an outer surface that is adapted to seal against tissue of the LAA when the LAA is pulled down onto the outer surface of the proximal cap.

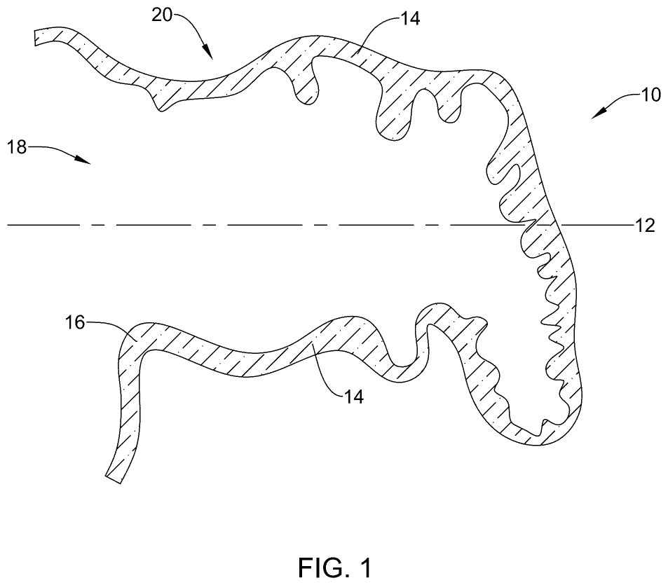

FIG. 1 is a partial cross-sectional view of a left atrial appendage 10. In some embodiments, the left atrial appendage (LAA) 10 may have a complex geometry and/or irregular surface area. It will be appreciated that the illustrated LAA 10 is merely one of many possible shapes and sizes for the LAA 10, which may vary from patient to patient. Those of skill in the art will also recognize that the medical devices, systems, and/or methods disclosed herein may be adapted for various sizes and shapes of the LAA 10, as necessary. The left atrial appendage 10 may include a generally longitudinal axis 12 arranged along a depth of a main body 20 of the left atrial appendage 10. The main body 20 may include a lateral wall 14 and an ostium 16 forming a proximal mouth 18. In some examples, a lateral extent of the ostium 16 and/or the lateral wall 14 may be smaller or less than a depth of the main body 20 along the longitudinal axis 12, or a depth of the main body 20 may be greater than a lateral extent of the ostium 16 and/or the lateral wall 14. In some examples, the LAA 10 may narrow quickly along the depth of the main body 20 or the left atrial appendage may maintain a generally constant lateral extent along a majority of the depth of the main body 20. In some examples, the LAA 10 may include a distalmost region formed or arranged as a tail-like element associated with a distal portion of the main body 20. In some examples, the distalmost region may protrude radially or laterally away from the longitudinal axis 12.

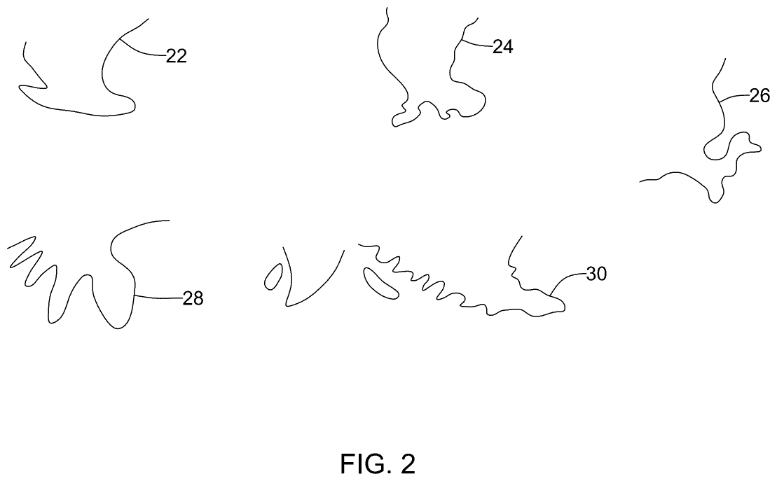

FIG. 2 provides several examples of particular LAA shapes that differ from that shown in FIG. 1. In some cases, these particular LAA shapes may be problematic for implanting an LAAC device because these shapes tend to be shallow. An LAA 22 is an example of a left atrial appendage having a shallow profile. An LAA 24 is an example of a left atrial appendage having a bi-lobed or whale tail-shaped profile. An LAA 26 is an example of a left atrial appendage having a shallow vertical chicken wing-shaped anatomy. In some cases, the LAA 26 may be considered as having a seahorse-shaped profile. An LAA 28 is an example of a left atrial appendage including trabeculations, or having a broccoli-shaped profile. An LAA 30 is an example of a left atrial appendage that has a wide profile, with posterior sloping trabeculations extending above an implant plane. It will be appreciated that these are merely illustrative, as other types and shapes of left atrial appendages are known. The illustrative LAAC devices descried herein may be used in any of a variety of different-shaped LAAs, including the LAA 10, the LAA 22, the LAA 24, the LAA 26, the LAA 28 and the LAA 30.

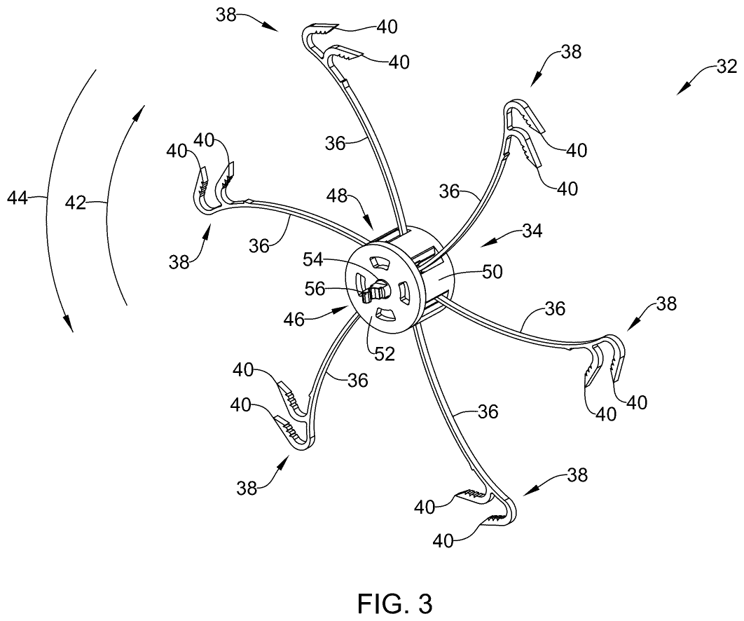

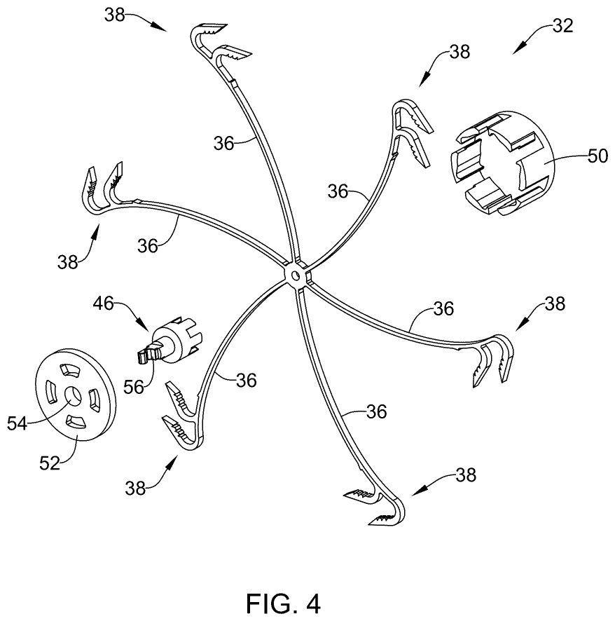

FIG. 3 is a perspective view of an illustrative LAAC device 32 that may be used in closing any of the LAA 10, the LAA 22, the LAA 24, the LAA 26, the LAA 28 and the LAA 30. FIG. 4 is an exploded perspective view of the illustrative LAAC device 32. The LAAC device 32 includes a central hub 34 and a plurality of engagement arms 36 that are coupled to the central hub 34. As shown, the LAAC device 32 includes a total of six engagement arms 36. This is merely illustrative, as the LAAC device 32 may include only three, four or five engagement arms 36. In some cases, the LAAC device 32 may include seven, eight, nine, ten or more engagement arms 36. It will be appreciated that there may be a tradeoff between the ability to grasp tissue within the LAA 10, the LAA 22, the LAA 24, the LAA 26, the LAA 28 and the LAA 30 and packing constraints for delivering the LAAC device 32 to the LAA 10, the LAA 22, the LAA 24, the LAA 26, the LAA 28 or the LAA 30. More engagement arms 36 provide additional capacity to grasp tissue, but may be more bulky when packed for delivery. Conversely, fewer engagement arms 36 may be better for packing, but may not provide as many opportunities to grasp tissue.

At least some of the engagement arms 36 include a tissue engaging region 38. In some cases, all of the engagement arms 36 include a tissue engaging region 38. In some cases, the tissue engaging region 38 may include one or more hooks 40. As shown, each of the tissue engaging regions 38 include a pair of hooks 40. At least some of the tissue engaging regions 38 may include only one hook 40. At least some of the tissue engaging regions 38 may include three or more hooks 40. In some cases, the one or more hooks 40 may be similarly angled along the engagement arm 36. In some cases, the one or more hooks 40 may be angled offset from one another along the engagement arm 36. For example, in an embodiment, one hook 40 may be offset from another hook 40 along an axis of the engagement arm 36. One hook 40 may be offset in a first direction that is orthogonal to a first circumferential direction indicated by an arrow 42, and another hook 40 may be offset in a second, opposite, direction that is orthogonal to the first circumferential direction indicated by the arrow 42. In some cases, a hook 40 may be offset as much as 60 degrees from the axis of the engagement arm 36. In some cases, each of the one or more hooks 40 on an engagement arm 36 may be offset in the same direction that is orthogonal to the first circumferential direction indicated by the arrow 42. There may be a similar tradeoff between tissue grasping and packing considerations with the hooks 40, similar to the number of engagement arms 36.

In some cases, the engagement arms 36 and the central hub 34 may be formed as an integral shape memory frame. In some cases, the engagement arms 36, including the tissue engaging regions 38, and the central hub 34 may be laser cut from a single piece of metal. As an example, the engagement arms 36 and the central hub 34 may be a Nitinol frame. In some cases, each of the engagement arms 36 may have the same length. In some cases, at least some of the engagement arms 36 may have different lengths. In some cases, having all of the engagement arms 36 with the same length can be helpful with closure of the LAA 10, the LAA 22, the LAA 24, the LAA 26, the LAA 28 or the LAA 30. In some cases, having different lengths can help with closure of the LAA 10, the LAA 22, the LAA 24, the LAA 26, the LAA 28 or the LAA 30.

It will be appreciated that the LAAC device 32 shown in FIGS. 3 and 4 may be considered as being in a configuration that corresponds to a configuration after the LAAC device 32 has been delivered but before the LAAC device 32 has contacted or substantially contacted tissue within the LAA 10, the LAA 22, the LAA 24, the LAA 26, the LAA 28 or the LAA 30. Prior to tissue engagement, as shown, at least some of the engagement arms 36 extend radially outwardly from the central hub 34. In some cases, at least some of the engagement arms 36 may be adapted to bend in a second circumferential direction that is opposite that of the first circumferential direction. The second circumferential direction is indicated by an arrow 44. In some cases, once the engagement arms 36 have engaged tissue, at least some of the engagement arms 36 may be adapted to bend further in the second circumferential direction.

The LAAC device 32 includes a drive mechanism 46 that is adapted to be engaged with the central hub 34. The drive mechanism 46 is adapted to rotate the central hub 34 in the first circumferential direction, as indicated by the arrow 42. In some cases, the drive mechanism 46 may be adapted to be releasably secured to a delivery device (not shown in FIG. 3 or 4). As an example, the drive mechanism 46 may form half of a train coupler-style connector, and a distal end of a delivery device may form the other half of a train coupler-style connector. A sheath over the two halves of the train coupler-style connector hold the two halves together such that the delivery device may be used to advance the LAAC device 32 to a desired position within the LAA 10, the LAA 22, the LAA 24, the LAA 26, the LAA 28 or the LAA 30 and to actuate the LAAC device 32 within the LAA 10, the LAA 22, the LAA 24, the LAA 26, the LAA 28 or the LAA 30.

In some cases, particularly if there is a desired to disengage the tissue of the LAA 10, the LAA 22, the LAA 24, the LAA 26, the LAA 28 or the LAA 30 in order to reposition the LAAC device 32, the drive mechanism 46 may also be adapted to rotate the central hub 34 in the second circumferential direction, as indicated by the arrow 44. In some instances, the drive mechanism 46 may be adapted to be releasably coupled to a delivery device that may be used to advance the LAAC device 32 into the LAA 10, the LAA 22, the LAA 24, the LAA 26, the LAA 28 or the LAA 30 and to then rotate the central hub 34. As the central hub 34 rotates in the first circumferential direction indicated by the arrow 42, and as the tissue engaging regions 38 engage into the tissue of the LAA 10, the LAA 22, the LAA 24, the LAA 26, the LAA 28 or the LAA 30, the engagement arms 36 will bend further in the second circumferential direction indicated by the arrow 44 and the tissue engaging regions 38 will thus pull the tissue of the LAA 10, the LAA 22, the LAA 24, the LAA 26, the LAA 28 or the LAA 30 down onto itself and down onto the LAAC device 32. If the central hub 34 is rotated far enough, the LAA 10, the LAA 22, the LAA 24, the LAA 26, the LAA 28 or the LAA 30 will essentially be closed off.

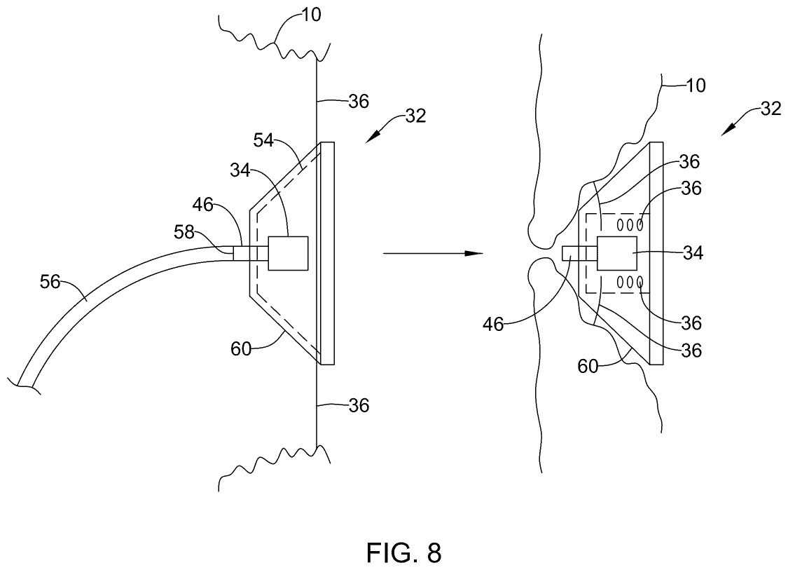

The LAAC device 32 may be considered as including a proximal cap 48. In some cases, as shown, the proximal cap 48 may have a cylindrical profile, and may include a cylindrical sleeve 50 and a proximal face 52. The proximal face 52 may include a centrally located aperture 54 that is adapted to allow a coupling surface 56 of the drive mechanism 46 to extend through the proximal face 52. In some cases, the proximal cap 48 may have a non-cylindrical profile. FIGS. 8 through 10, which will be discussed, include examples of non-cylindrical profiles for the proximal cap 48. In some cases, when the tissue of the LAA 10, the LAA 22, the LAA 24, the LAA 26, the LAA 28 or the LAA 30 is pulled down on itself, the tissue contacts the proximal cap 48. In some cases, the proximal cap 48 helps to seal against the tissue.

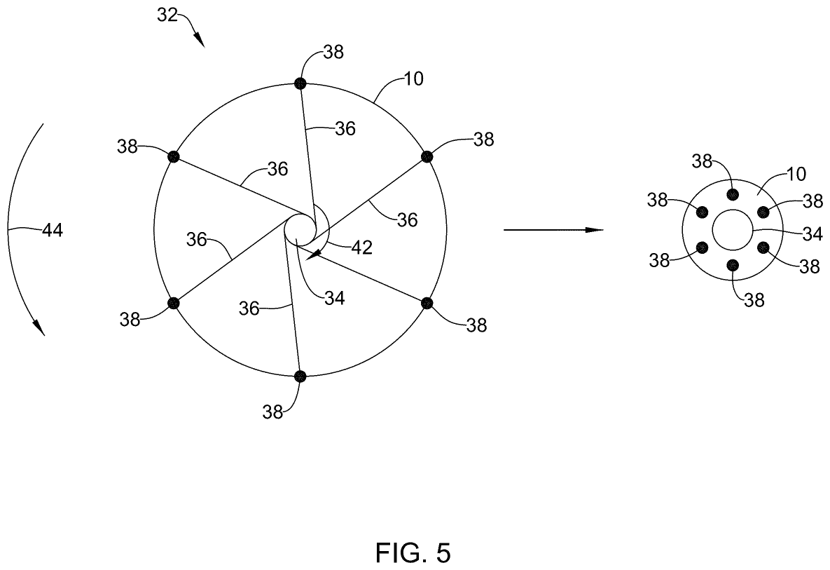

FIG. 5 provides a schematic illustration of how the LAAC device 32 functions to close the LAA 10. While the LAA 10 is labeled, it will be appreciated that any of the other LAAs including the LAA 22, the LAA 24, the LAA 26, the LAA 28 or the LAA 30 are equally applicable. As seen on the left side of FIG. 5, the tissue engaging regions 38 have engaged the tissue of the LAA, which in this particular example is the LAA 10. Rotation of the central hub 34 in the first circumferential direction indicated by the arrow 42 has begun. By continuing to rotate the central hub 34 in the first circumferential direction, the engagement arms 36 will continue to bend back over in the second circumferential direction indicated by the arrow 44, and the tissue of the LAA 10 will be pulled increasingly closer to the LAAC device 32. Eventually, as seen on the right hand side of FIG. 5, the tissue of the LAA 10 has collapsed down onto the LAAC device 32. Collapsing the LAA 10 down on itself in this manner closes the LAA 10. Closing the LAA 10 means that no blood or virtually no blood will flow into or out of the LAA 10, reducing the possibilities of any emboli exiting the LAA 10 and passing into circulation.

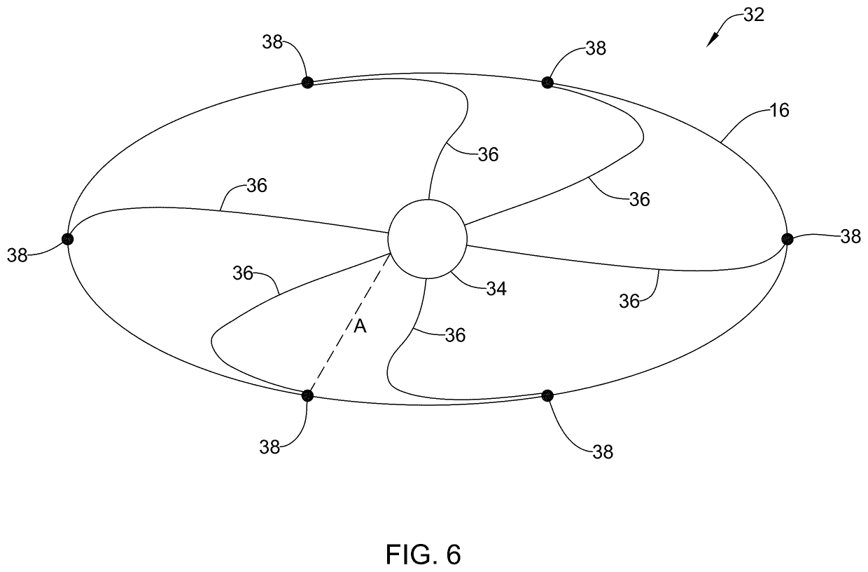

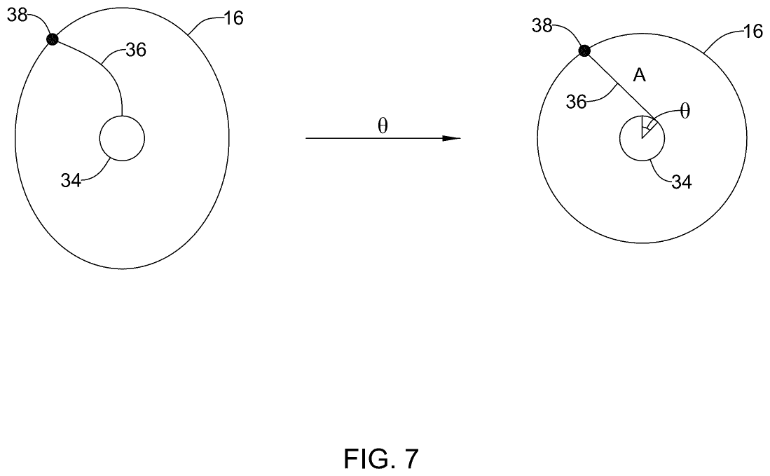

In some cases, the LAA 10, the LAA 22, the LAA 24, the LAA 26, the LAA 28 or the LAA 30, or at least the ostium 16 thereof may not be circular. FIGS. 6 and 7 show how the LAAC device 32 adapts to a non-circular ostium 16. While the ostium 16 was introduced as being part of the LAA 10, it will be appreciated that the ostium 16 may be part of any of the other LAAs including the LAA 22, the LAA 24, the LAA 26, the LAA 28 or the LAA 30. In the example shown, the ostium 16 is ovoid in shape, meaning that the ostium 16 has a longer diameter in one direction and a shorter diameter in an orthogonal second direction. As illustrated, the horizontal (in the illustrated orientation) diameter is greater than the vertical diameter. The engagement arms 36 that extend to the left side of the ostium 16 and to the right side of the ostium 16 are not as doubled over as the engagement arms 36 that extend towards the top of the ostium 16 and towards the bottom of the ostium 16. The tissue engaging region 38 of each of the engagement arms 36 have engaged tissue.

A particular tissue engaging region 38 is engaged with the tissue of the ostium 16 such that a straight-line dimension between the tissue engaging region 38 and the central hub 34 is indicated as “A”, even though the particular engagement arm 36 has an overall length that is greater than the dimension “A”. Rotating the central hub 34 in the first circumferential direction a distance θ (theta) results in the particular engagement arm 36 having an effective length of “A”, as shown in FIG. 7. The distance θ (theta) may be considered a rotational direction, for example, and may be expressed in degrees or radians. The greater the difference between the overall length of the particular engagement arm 36 and a straight line distance between the tissue engaging region 38 (once engaged) and the central hub 34 (shown as the distance “A”), the greater that the rotational distance θ (theta) may be.

FIGS. 8, 9 and 10 are schematic illustrations showing how the LAA 10 is drawn down or collapsed onto several different example proximal caps. While the LAA 10 is labeled, it will be appreciated that any of the other LAAs including the LAA 22, the LAA 24, the LAA 26, the LAA 28 or the LAA 30 are equally applicable. FIG. 8 shows a proximal cap 54 that has a frustoconical shape. The proximal cap 54 may be considered as being an example of the proximal cap 48. On the left hand side of FIG. 8, the LAAC device 32 is shown coupled with a delivery device 56 having a distal end 58 that is releasably coupled with the drive mechanism 46. On the right hand side of FIG. 8, the LAAC device 32 has been actuated, and as a result the tissue of the LAA 10 has been collapsed down onto an outer surface 60 of the proximal cap 54. The delivery device 56 has been removed. As a result of pulling the tissue of the LAA 10 down onto the proximal cap 54, it will be appreciated that the LAAC device 32 is largely captured within the tissue of the LAA 10. This means that the LAAC device 32 is substantially removed from any significant blood flow. This can be beneficial in reducing changes for DRT (device related thrombosis).

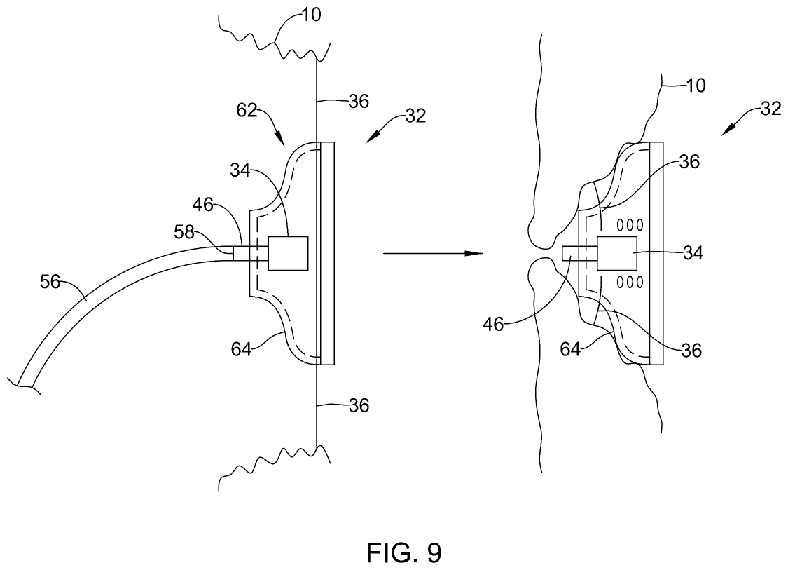

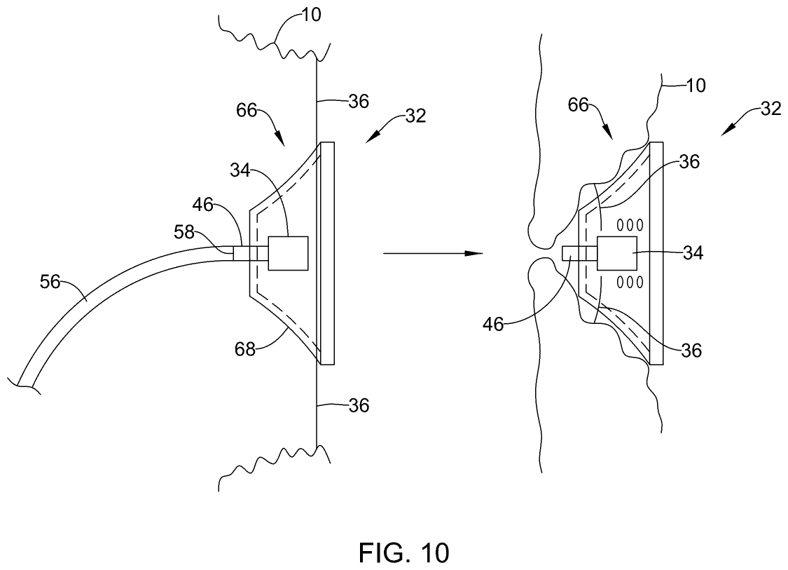

In FIG. 9, the LAAC device 32 has a proximal cap 62 having a curved outer surface 64. The proximal cap 62 may be considered as being an example of the proximal cap 48. The tissue of the LAA 10 will pull down onto the curved outer surface 64, as shown on the right hand side of FIG. 9. In FIG. 10, the LAAC device 32 has a proximal cap 66 having a concave outer surface 68. The proximal cap 66 may be considered as being an example of the proximal cap 48. The tissue of the LAA 10 will pull down onto the concave outer surface 68, as shown on the right hand side of FIG. 10. Other shapes and profiles for the proximal cap 48 are also contemplated.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear-elastic” or “non-super-elastic” which, although may be similar in chemistry to conventional shape memory and super elastic varieties, may exhibit distinct and useful mechanical properties. Linear elastic and/or non-super-elastic nitinol may be distinguished from super-elastic nitinol in that the linear elastic and/or non-super-elastic nitinol does not display a substantial “super-elastic plateau” or “flag region” in its stress/strain curve like super-elastic nitinol does. Instead, in the linear-elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super-elastic plateau and/or flag region that may be seen with super-elastic nitinol. Thus, for the purposes of this disclosure linear-elastic and/or non-super-elastic nitinol may also be termed “substantially” linear-elastic and/or non-super-elastic nitinol.

In some cases, linear-elastic and/or non-super-elastic nitinol may also be distinguishable from super-elastic nitinol in that linear-elastic and/or non-super-elastic nitinol may accept up to about 2-5% strain while remaining substantially elastic (e.g., before plastically deforming) whereas super-elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear-elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear-elastic and/or non-super-elastic nickel-titanium alloy. The mechanical bending properties of such material may therefore be generally inert to the effect of temperature over this very broad range of temperature. In some embodiments, the mechanical bending properties of the linear-elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear-elastic and/or non-super-elastic nickel-titanium alloy maintains its linear-elastic and/or non-super-elastic characteristics and/or properties.

In some embodiments, the linear-elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, the devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

A sheath or covering (not shown) may be disposed over portions or all of the devices described herein in order to define a generally smooth outer surface. In other embodiments, however, such a sheath or covering may be absent. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A left atrial appendage closure (LAAC) device adapted to pull a left atrial appendage (LAA) closed on itself, the LAAC device comprising:
a central hub;
a plurality of engagement arms coupled to the central hub, at least some of the plurality of engagement arms including a tissue engaging region, the tissue engaging region including one or more hooks extending in a first circumferential direction; and
a drive mechanism engaged with the central hub and adapted to rotate the central hub in the first circumferential direction.

2. The LAAC device of claim 1, wherein prior to engagement with tissue of the LAA, at least some of the plurality of engagement arms have an expanded, post-delivery configuration in which at least some of the plurality of engagement arms extend radially outwardly from the central hub.

3. The LAAC device of claim 2, wherein prior to engagement with tissue of the LAA, the plurality of engagement arms have an expanded post-delivery configuration in which each of the engagement arms are adapted to bend in a second circumferential direction that is opposite of the first circumferential direction.

4. The LAAC device of claim 3, wherein after engagement with tissue of the LAA, the plurality of engagement arms are each adapted to bend further in the second circumferential direction.

5. The LAAC device of claim 1, further comprising a proximal cap extending over the central hub and the drive mechanism, the proximal cap including an outer surface.

6. The LAAC device of claim 5, wherein the outer surface of the proximal cap is adapted to seal against tissue of the LAA when the LAA is pulled down onto the outer surface of the proximal cap.

7. The LAAC device of claim 1, wherein the plurality of engagement arms and the central hub are formed as an integral shape memory frame.

8. The LAAC device of claim 7, wherein the integral shape memory frame comprises a Nitinol frame.

9. The LAAC device of claim 1, wherein the drive mechanism is adapted to be releasably secured to a delivery device.

10. The LAAC device of claim 1, wherein each of the plurality of engagement arms are the same length.

11. A left atrial appendage closure (LAAC) device adapted to pull a left atrial appendage (LAA) closed on itself, the LAAC device comprising:
a central hub; and
a plurality of engagement arms that, prior to engagement with tissue of the LAA, have an expanded, post-delivery configuration in which each of the plurality of engagement arms extend radially outwardly from the central hub;
at least some of the plurality of engagement arms including one or more hooks within a distal region thereof extending in a first circumferential direction.

12. The LAAC device of claim 11, further comprising a drive mechanism engaged with the central hub and adapted to rotate the central hub in the first circumferential direction.

13. The LAAC device of claim 11, wherein prior to engagement with tissue of the LAA, the plurality of engagement arms are each adapted to bend in a second circumferential direction that is opposite of the first circumferential direction.

14. The LAAC device of claim 11, wherein after engagement with tissue of the LAA, the plurality of engagement arms are each adapted to bend further in the second circumferential direction.

15. The LAAC device of claim 11, wherein the plurality of engagement arms and the central hub are formed as an integral Nitinol frame.

16. The LAAC device of claim 11, further comprising a proximal cap extending over the central hub, the proximal cap including an outer surface that is adapted to seal against tissue of the LAA when the LAA is pulled down onto the outer surface of the proximal cap.

17. A left atrial appendage closure (LAAC) device adapted to pull a left atrial appendage (LAA) closed on itself, the LAAC device comprising:
a central hub;
a drive mechanism engaged with the central hub;
a plurality of engagement arms having a configuration in which each of the plurality of engagement arms extend radially outwardly from the central hub in the absence of applied forces;
at least some of the plurality of engagement arms including one or more hooks within a distal region thereof extending in a first circumferential direction.

18. The LAAC device of claim 17, wherein the plurality of engagement arms are each adapted to bend further in the second direction after the one or more hooks engage tissue.

19. The LAAC device of claim 17, wherein the plurality of engagement arms and the central hub comprise a Nitinol frame.

20. The LAAC device of claim 17, further comprising a proximal cap extending over the central hub, the proximal cap including an outer surface that is adapted to seal against tissue of the LAA when the LAA is pulled down onto the outer surface of the proximal cap.
